(19) United States
(12) Patent Application Publication
(10) Pub. No.: US 2020/0316279 A1
(43) Pub. Date: Oct. 8, 2020

(54) AIR MANAGEMENT DEVICE FOR DIALYSIS MACHINES

(71) Applicant: FRESENIUS MEDICAL CARE HOLDINGS, INC., Waltham, MA (US)

(72) Inventors: Kulwinder S. Plahey, Martinez, CA (US); James J. Peterson, Benicia, CA (US)

(21) Appl. No.: 16/372,445

(22) Filed: Apr. 2, 2019

(51) Int. Cl. A61M 1/28; A61M 1/36

(52) U.S. Cl. CPC A61M 1/288; A61M 1/282; A61M 2205/3334; A61M 1/3643; A61M 2205/3379; A61M 1/3627

(57) ABSTRACT

A dialysis system may include a dialysis machine (e.g., a peritoneal dialysis machine or a hemodialysis machine) for transferring a liquid to a patient via tubing. The dialysis system may include an air management device for filtering out air content from the liquid prior to transferring to the patient. The air management device may be a connector cap for use in a peritoneal dialysis system during a priming process. Alternatively, the air management device may be a drip chamber for use in a hemodialysis system. The air management device may include a vent. In addition, the air management device may include a movable element for sealing the vent. In addition, and/or alternatively, the air management device may include a reservoir for receiving an overflow of liquid.

### FIELD

The disclosure generally relates to dialysis machines and/or systems, and more particularly to air management in dialysis machines, systems, and methods.

### BACKGROUND

Dialysis machines are known for use in the treatment of renal disease. The two principal dialysis methods are hemodialysis (HD) and peritoneal dialysis (PD). During hemodialysis, the patient's blood is passed through a dialyzer of a hemodialysis machine while also passing dialysate through the dialyzer. A semi-permeable membrane in the dialyzer separates the blood from the dialysate within the dialyzer and allows diffusion and osmosis exchanges to take place between the dialysate and the blood stream. During peritoneal dialysis, the patient's peritoneal cavity is periodically infused with dialysate or dialysis solution. The membranous lining of the patient's peritoneum acts as a natural semi-permeable membrane that allows diffusion and osmosis exchanges to take place between the solution and the blood stream. Automated peritoneal dialysis machines, called APD cyclers, are designed to control the entire peritoneal dialysis process so that it can be performed at home, usually overnight, without clinical staff in attendance.

A dialysis machine, such as a peritoneal dialysis machine, may include one or more containers (e.g., bags) containing a fluid, e.g., a dialysate, for patient infusion. In peritoneal dialysis machines, for example, tubing is connected to a peritoneal catheter which has been inserted into an abdomen of a patient for flowing fresh dialysate and removing used dialysate, waste, and excess fluid. Prior to patient insertion and a dialysis treatment, the tubing is primed with dialysate to minimize air in the tubing being delivered to the peritoneal cavity of the patient, which may cause cramps or discomfort.

It is with respect to these and other considerations that the present improvements may be useful.

### SUMMARY

This Summary is provided to introduce a selection of concepts in a simplified form that are further described below in the Detailed Description. This Summary is not intended to necessarily identify key features or essential features of the claimed subject matter, nor is it intended as an aid in determining the scope of the claimed subject matter.

According to an exemplary embodiment of the present disclosure, a dialysis system for conducting a dialysis treatment is disclosed. The dialysis system may include a dialysis machine for transferring a liquid to a patient via tubing and a connector cap removably coupled to an end of the tubing, the connector cap being arranged and configured to filter out air content prior to transferring the liquid to the patient.

In this and other embodiments, the connector cap includes a first open end and a second end, the second end including a vent.

In this and other embodiments, the end of the tubing is insertable into the first open end of the connector cap, the air content being filterable out of the vent of the second end of the connector cap.

In this and other embodiments, the connector cap includes a hydrophobic filter disposed at the second end of the connector cap, the air content being filterable out of the vent through the hydrophobic filter.

In this and other embodiments, the connector cap includes a circumferential flange extending from an inner surface of the connector cap, the flange including an aperture formed therein, the aperture defining a fluid flow path for the liquid, or air content, or both, from the first end to the second end through the connector cap.

In this and other embodiments, the connector cap includes a movable element, the movable element being movably positioned between the flange and the second end of the connector cap.

In this and other embodiments, the movable element allows passage of the air content flowing through the connector cap.

In this and other embodiments, in response to the liquid entering the connector cap, the movable element is movable by the liquid to seal the vent.

In this and other embodiments, the connector cap includes a reservoir for receiving the liquid.

In this and other embodiments, the dialysis machine is a peritoneal dialysis machine.

In this and other embodiments, the liquid is a dialysate solution.

According to another exemplary embodiment of the present disclosure, a method of conducting a dialysis treatment is disclosed. The method may include transferring a liquid to a patient via tubing by a dialysis machine, removably coupling a connector cap to an end of the tubing, the connector cap including a vent, and filtering out air content via the vent of the connector cap prior to transferring the liquid to the patient.

In this and other embodiments, the connector cap includes an open first end and a second end, the vent being disposed in the second end.

In this and other embodiments, the connector cap includes a hydrophobic filter disposed at the second end of the connector cap, such that the air content is filtered out of the vent through the hydrophobic filter.

In this and other embodiments, the connector cap includes a circumferential flange extending from an inner surface of the connector cap, the flange including an aperture defining a fluid flow path, and a movable element movably positioned between the flange and the second end of the connector cap, wherein, in response to a liquid flowing into the connector cap, sealing the vent by the movable element.

According to another exemplary embodiment of the present disclosure, a connector cap for a dialysis system is disclosed. The connector cap may include a hollow body having a first open end and a second end, the second end having a vent, a flange extending inward from an inner surface of the hollow body, and a movable element disposed in the second end of the hollow body, the movable element being movable positioned between the flange and the second end of the hollow body.

In this and other embodiments, the tubing is insertable in the first open end of the hollow body of the connector cap.

In this and other embodiments, the connector cap may further include a reservoir extending from the hollow body, such that the reservoir is substantially parallel to the tubing.

In this and other embodiments, the dialysis system is a peritoneal dialysis system.

In this and other embodiments, the moveable element is configured to move within the connector cap to seal the vent in the second end of the hollow body, or to seal an aperture in the flange, depending on the flow path of fluid through the connector cap.

In this and other embodiments, the fluid is liquid, or air content, or both.

According to another exemplary embodiment of the present disclosure, a dialysis system for conducting a dialysis treatment is disclosed. The dialysis system may include a dialysis machine for circulating a liquid including blood into and out of a patient via tubing, and an air managed drip chamber coupled to an end of a first tubing for receiving the liquid and a second tubing for transferring the liquid to the patient, the drip chamber including a body having a cavity for receiving the liquid therein, a vent for releasing air content from the cavity prior to transferring the liquid to the patient; and a movable element positioned within the cavity, the movable element being arranged and configured to seal the vent when an amount of liquid is received within the cavity.

In this and other embodiments, the drip chamber includes a body having a first end and a second end, the first tubing being receiving within the first end of the body, the second tubing being received within the second end of the body, and the vent is formed in the first end of the body.

In this and other embodiments, the body includes an internal partition extending into the cavity for creating a contained channel for the moveable element.

In this and other embodiments, the movable element is arranged and configured to float on the liquid received within the cavity, the moveable element contacting and sealing the vent when a sufficient amount of liquid is received within the cavity.

### BRIEF DESCRIPTION OF THE DRAWINGS

By way of example, specific embodiments of the disclosed machine will now be described, with reference to the accompanying drawings, in which:

FIG. 1A illustrates a perspective view of an exemplary embodiment of an air management device (e.g., a connector cap) in accordance with the present disclosure;

FIGS. 1B-1D illustrate section views of the air management device (e.g., the connector cap) of FIG. 1A connecting with tubing;

FIGS. 2A-2B illustrate section views of alternate exemplary embodiments of air management devices (e.g., connector caps) in accordance with the present disclosure;

FIG. 3 illustrates a section view of another exemplary embodiment of an air management device (e.g., an air managed drip chamber) in accordance with the present disclosure;

FIG. 4A illustrates an exemplary embodiment of a dialysis machine in accordance with the present disclosure;

FIG. 4B illustrates an exemplary embodiment of a dialysis machine in accordance with the present disclosure;

FIG. 5 illustrates another exemplary embodiment of a dialysis machine in accordance with the present disclosure;

FIG. 6 illustrates an exemplary embodiment a dialysis machine in accordance with the present disclosure; and

FIG. 7 illustrates an exemplary embodiment of a dialysis machine in accordance with the present disclosure.

### DETAILED DESCRIPTION

The present embodiments will now be described more fully hereinafter with reference to the accompanying drawings, in which several exemplary embodiments are shown. The subject matter of the present disclosure, however, may be embodied in many different forms and should not be construed as limited to the embodiments set forth herein. Rather, these embodiments are provided so that this disclosure will be thorough and complete, and willfully convey the scope of the subject matter to those skilled in the art. In the drawings, like numbers refer to like elements throughout.

As described above, in a dialysis operation, tubing may be connected between a dialysis machine and a patient for delivering liquid into the patient's body. For example, in peritoneal dialysis operations, tubing is connected between a dialysis machine and a patient for delivering fresh dialysate into the patient's peritoneal cavity, and removing used dialysate and contaminants after a predetermined time. A patient may undergo several cycles of delivering a fresh batch of dialysate and removing the used dialysate and contaminants in a single treatment. In some embodiments, a peritoneal dialysis treatment may be performed at home, and may occur overnight while a patient is sleeping.

In bags containing fresh dialysate, an amount of air may also be present, for example, due to fill levels, osmosis, and/or other conditions. Each bag may contain a quantity of air, which may be present as a result of the bag being not completely filled with liquid during manufacture. Additionally, bags may be stored for a period of time prior to sale and/or use by a patient, e.g., 1-2 years or longer. Certain bag materials may be more susceptible to osmosis, for example, a Biofine™ material bag may have a greater amount of air after a period of storage than a bag made of a different material, such as a polyvinyl chloride (PVC) material. For example, a bag may contain a range of approximately 20 cc to 150 cc of air. If the dialysis machine draws a combination of dialysate and air (e.g., air bubbles) from one of the bags or elsewhere in the system, the dialysis machine may deliver less than the prescribed volume of dialysate to the patient over the course of the treatment and/or a potentially painful build-up of excess air in the patient may result. For example, air delivered to the patient may result in the patient experiencing discomfort, such as shoulder or abdominal pain. To minimize discomfort so that a patient may be able to sleep through the treatment, tubing extending between the dialysis machine and the patient may be primed with dialysate prior to the peritoneal dialysis treatment.

Although the term “bag” is used throughout, it should be understood that a bag may be any type of container capable of holding a liquid, e.g., a dialysate. In some embodiments, a container may include a container in which dry concentrates are mixed with water to generate dialysate suitable for a dialysis treatment.

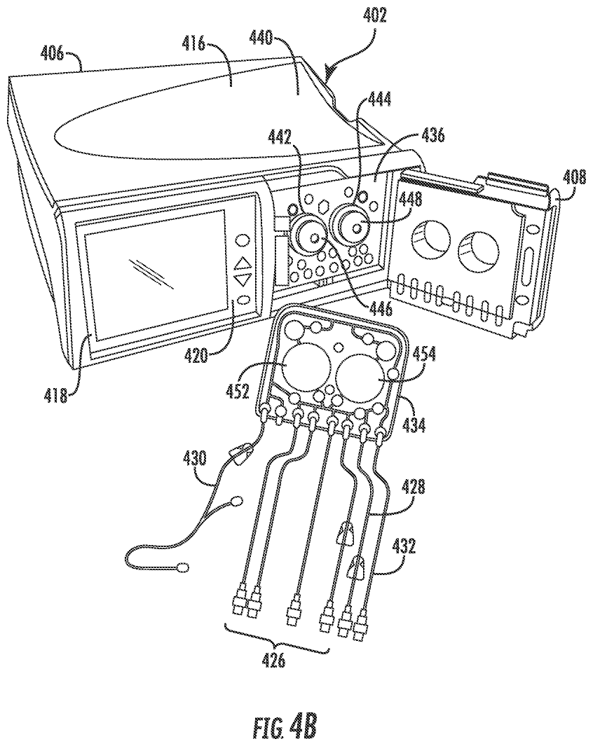

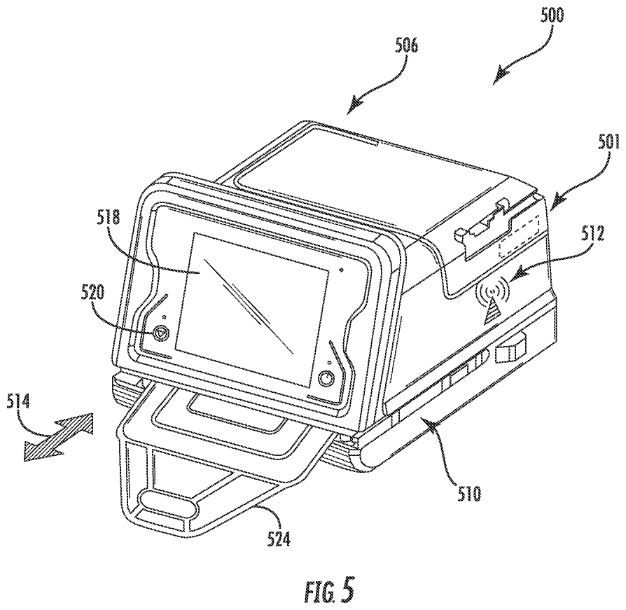

In Automated Peritoneal Dialysis (APD) systems, such as, for example, those shown in FIGS. 4A and 5, a cassette or tubing set, such as, for example, as shown in FIG. 4B may be used. At treatment start-up, the cassette or tubing system normally contains air, which must be purged or primed prior to connecting to the patient and infusing the fresh dialysate into the patient.

In connection with a peritoneal dialysis operation, tubing is primed when a liquid (e.g., dialysate) is flowed through the tubing prior to being inserted in the patient to minimize, or eliminate, air content present in the tubing. As described above, priming may minimize or prevent air infusion to the peritoneal cavity of the patient, thereby minimizing potential pain, cramps, and/or other discomfort during the dialysis treatment. However, verification of full priming of the tubing may be challenging. A patient may have difficulty manually checking the full length of the tubing as the fluid (e.g., dialysate) may be clear (e.g., transparent), the tubing may be several feet long and may have variations in length, and/or several additional steps may be needed to set up the dialysis machine for treatment. Additionally, since priming of the tubing (e.g., patient line) may occur prior to being connected to, for example, a patient's catheter, the end of the tubing may be opened during priming. If the patient does not fully prime the tubing prior to connection to the patient's catheter, air may remain in the tubing during any subsequent treatment procedure, and may be delivered into the patient's peritoneal cavity, thereby causing discomfort. In some instances, the patient may not notice when priming is completed, e.g., when the air has been fully pushed out and dialysate is flowing out the end of the tubing, so that dialysate may overflow out of the end of the tubing resulting in a messy condition and wasting dialysate. An unacceptable amount of wasted dialysate may result in the patient not receiving a full prescribed treatment or a treatment time being unnecessarily extended. When a patient receives less than 90% of a dialysate treatment, it may be considered ineffective.

In accordance with one aspect of the present disclosure, an air management device may be used to filter out air content in a liquid before introduction of the liquid into the patient's body. For example, in a peritoneal dialysis operation, in accordance with one aspect of the present disclosure, the air management device may be in the form of a connector cap. In use, the connector cap may be removably connectable to an end of the tubing (e.g., the patient line) prior to connecting to the patient's catheter. In use, the connector cap facilitates filtering out air content in a liquid such as dialysate.

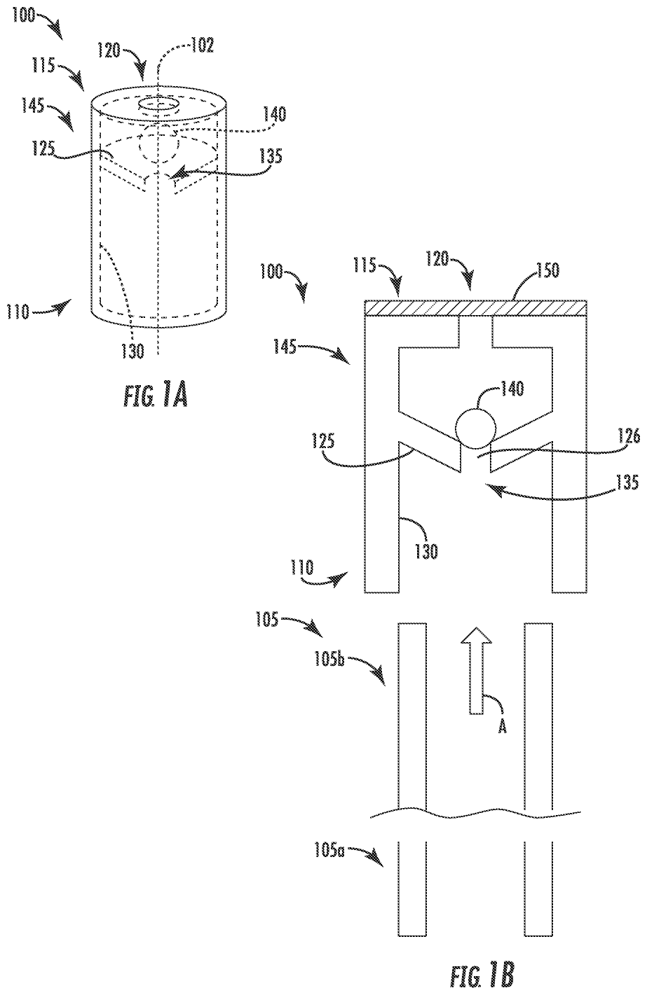

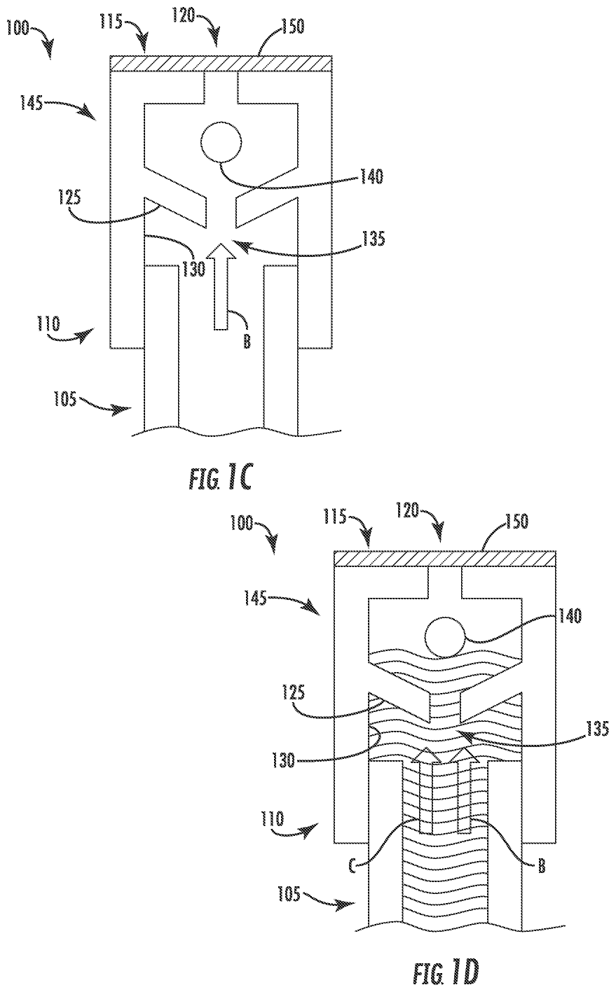

Referring now to FIGS. 1A-1D, an example embodiment of the air management device (e.g., the connector cap) 100 in accordance with the present disclosure is shown. The connector cap 100 may be removably coupled, attached, etc. (used interchangeably herein without the intent to limit) to the tubing 105 (e.g., the patient line) during a priming step prior to a dialysis treatment. During use, the liquid (e.g., dialysate) may flow through the tubing 105 from a first end 105a of the tubing 105 coupled to, for example, a container such as, for example, a dialysate bag, to a second end 105b of the tubing 105. To perform the dialysis treatment, the second end 105b of the tubing 105 may be coupled to, for example, a patient's catheter (not shown). In accordance with one aspect of the present disclosure, prior to the treatment, the second end 105b of the tubing 105 may be coupled to the connector cap 100. The second end 105b of the tubing 105 and the connector cap 100 may be coupled together by any suitable mechanism now known or hereafter developed. For example, referring to FIG. 1B, the second end 105b of the tubing 105 and the connector cap 100 may be coupled together by moving the connector cap 100 over the second end 105b of the tubing 105 in a direction indicated by arrow “A.” The connector cap 100 may be held substantially vertical during priming by any suitable mechanism such as, for example, a holder, a bracket, etc., so that the second end 115 may be positioned vertically above the first end 110.

The connector cap 100 may be formed as a substantially cylindrical hollow body extending along a longitudinal axis 102, although it is envisioned that the connector cap 100 may be formed in any shape suitable for connection with the tubing 105. The connector cap 100 may have a first end 110 and a second end 115 opposite the first end 110. The first end 110 may be open (e.g., an open-ended enclosure), to receive at least a portion of the second end 105b of the tubing 105. For example, the connector cap 100 may be cylindrical so that the second end 105b of the tubing 105 may be insertable into the connector cap 100 for coupling the connector cap 100 to the tubing 105. In one embodiment, when connected to the second end 105b of the tubing 105, the connector cap 100 is arranged and configured to couple to the second end 105b of the tubing 105 in a seal-tight manner. For example, the connector cap 100 may be coupled to the second end 105b of the tubing 105 via a threaded connection, and/or other sealing mechanisms such as, for example, an O-ring, seal, or combinations thereof, may be utilized. Thus arranged, leaks at the connection between the connector cap 100 and the second end 105b of the tubing 105 may be minimized and/or prevented.

The second end 115 of the connector cap 100 may be formed as a flat surface, although in some embodiments the second end 115 may be formed as a frusto-conical portion, or any other shape extendable from the hollow body to define an enclosed second end. The second end 115 of the connector cap 100 may include a vent 120. The vent 120 may be disposed centrally in the second end 115 of the connector cap 100, e.g., along or coaxial with a longitudinal axis 102 of the connector cap 100. In one embodiment, the vent 120 may be a circular hole, e.g., a pinhole vent, although the vent 120 may be formed in any shape to allow for air content and/or other gases to escape from the connector cap 100 during priming.

The connector cap 100 may include a baffle, a flange, etc. 125 (used interchangeably herein without the intent to limit), extending inward from an inner surface 130 of the connector cap 100. The flange 125 may extend radially inward from an inner surface 130 of the connector cap 100 to define a fluid flow path 135 from the first end 110 through to the second end 115 of the connector cap 100. That is, as shown, the flange 125 may include an aperture 126 formed therein to define the fluid flow path 135. In use, as the tubing 105 is primed, air content, e.g., as indicated at arrow “B” in FIG. 1C, may be pushed out of the second end 105b of the tubing 105 into the first end 110 of the connector cap 100. Air content may flow from the first end 110 of the connector cap 100 through the fluid flow path 135 to the second end 115 of the connector cap 100 and out of the vent 120.

In some embodiments, the flange 125 may extend circumferentially around the inner surface 130 of the connector cap 100 as a single flange. In other embodiments, the flange 125 may be formed of multiple pieces extending from the inner surface 130, and may be staggered circumferentially and/or axially along the longitudinal axis 102. The flange 125 may extend from the inner surface 130 substantially perpendicular, e.g., approximately 90°±10°. Alternatively, in some embodiments, the flange 125 may extend from the inner surface 130 at an angle. In some embodiment, the flange may be integrally formed with the connector cap 100. Alternatively, the flange 125 may be separately formed and coupled thereto by any suitable manner including, for example, welding, adhesion, etc. In use, the flange 125 may have any suitable shape now known or hereafter developed and may be configured in any suitable manner to prevent the movable element 140, as will be described below, from passing through the aperture 126 formed in the flange 125. For example, the flange 125 can be flat, conical, etc. The flange 125 can be formed as a single piece or a series or fingers, projections, ribs, etc. extending from the inner surface 130.

The connector cap 100 may further include a movable element 140 disposed in an upper portion 145 of the connector cap 100 between the flange 125 and the second end 115 of the connector cap 100. The movable element 140 may be free to move in the upper portion 145, e.g., the movable element 140 may be bounded within the hollow body of the connector cap 100 between the second end 115 of the connector cap 100 and the flange 125 but remain otherwise unattached to the connector cap 100. The movable element 140 may be sized larger than the fluid flow path 135 (e.g., aperture 126) and the vent 120, so that the movable element 140 may be retained in the upper portion 145 of the connector cap 100.

The movable element 140 may be formed of a buoyant material, and may sit over the fluid flow path 135 when no fluid is moving through the connector cap 100 (e.g., angled surface of the flange 125 causes the movable element 140 to reside or be positioned over the aperture 126). In some embodiments, the movable element 140 may be colored to aid in visualization of movement. During use, when fluid, e.g., air content and/or liquid, flow through the connector cap from the first end 110 to the second end 115 of the connector cap 100, the movable element 140 may be pushed or moved away from the aperture 126 defined in the flange 125 and toward the vent 120. When priming has completed, the liquid (e.g., dialysate) may reach the connector cap 100, as indicated at arrow “C” shown in FIG. 1D. The movable element 140 may float on the liquid (e.g., dialysate) until reaching or contacting the second end 115 of the connector cap 100. At this point, the movable element 140 may be arranged and configured to mate or seal, the vent 120 formed in the connector cap 100. It is understood that the shapes of the vent 120 and the movable element 140 may be formed to mate together. For example, the movable element 140 may be formed as a rounded component such as a sphere or elliptical component, e.g., to mate and seal a circular vent 120. In other embodiments, the movable element 140 may be any shape, including but not limited to a disk and/or conical plug, to seal the vent 120. Sealing the vent 120 may reduce, minimize, and/or eliminate liquid (e.g., dialysate) leaking from the vent 120. When the movable element 140 seals the vent 120, the priming operation may be complete. In some embodiments, a user may manually indicate that the priming operation is complete. Alternatively, and/or in addition, in some embodiment, the dialysis machine may automatically determine that priming is complete. In some embodiments, the dialysis machine may alert the user when the vent 120 is sealed. The user may then disconnect the connector cap 100 from the tubing 105 and couple the tubing 105 to, for example, a patient catheter.

Additionally, and/or alternatively, the connector cap 100 may include a filter 150. The filter 150 may be any suitable filter such as, for example, a hydrophobic filter, so that the air content may flow through, but liquid such as the dialysate may be prevented by the filter 150 from exiting the connector cap 100. That is, for example, in one embodiment, the filter 150 may include a plurality of pores sized and configured to allow air to flow through the filter 150 but prevent liquid from flowing through the filter 150. One example of an embodiment of a filter that may be used is disclosed in U.S. Published Patent Application No. 2019/0076590 A1 to Plahey et al., entitled “Hydrophobic Filters for Air Management in Dialysis Machines,” which is incorporated herein by reference. The filter 150 may be formed as a flexible material, and may formed to the shape of the connector cap 100. In one embodiment, the filter 150 may be positioned on the outside of the connector cap 100 adjacent to the vent 120, although it is envisioned that the filter 150 may be positioned elsewhere including, for example, within the vent 120.

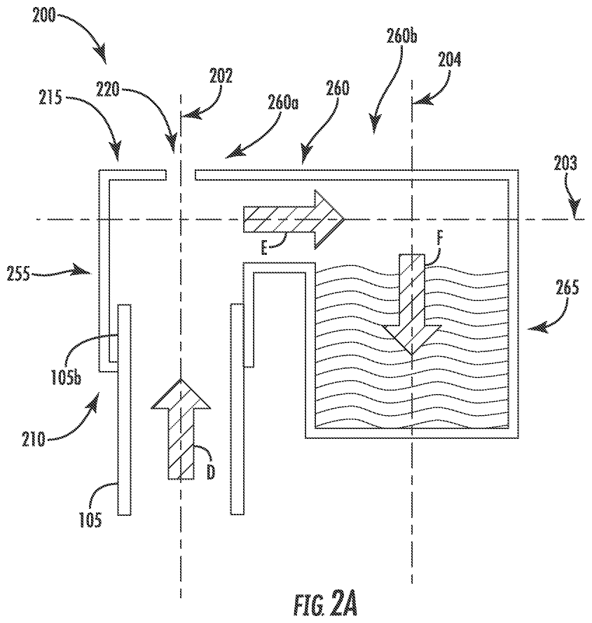

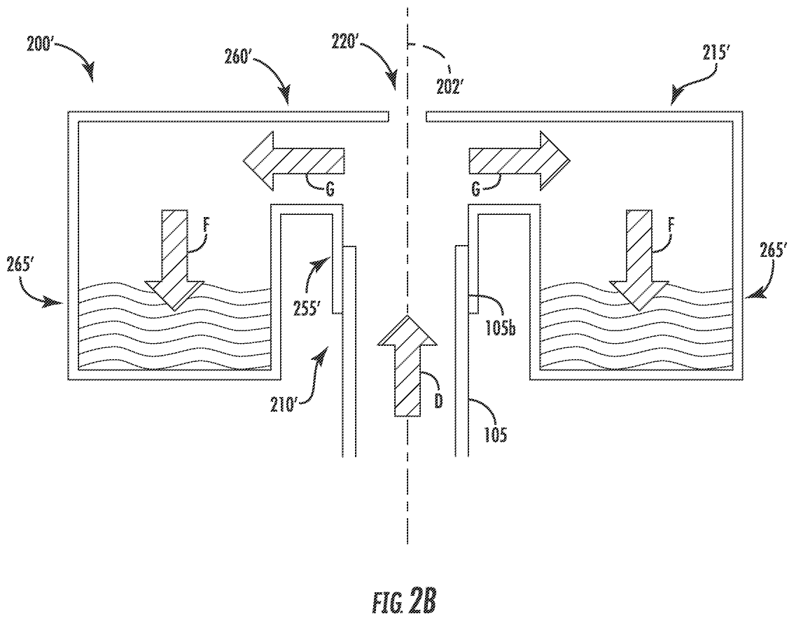

Referring now to FIGS. 2A-2B, alternate example embodiments of air management devices (e.g., connector caps) 200, 200′ in accordance with the present disclosure are shown. The connector cap 200, 200′ may be coupled to the second end 105b of the tubing 105. The connector cap 200, 200′ may be a hollow body having a first end 210, 210′ and a second end 215, 215′. The second end 215, 215′ may be positioned to be vertically above the first end 210, 210′. The connector cap 200, 200′ may include an optional vent 220, 220′ in the second end 215, 215′. In use, if incorporated, the vent 220, 220′ could be positioned anywhere along the second end 215, 215′.

The hollow body of the connector cap 200, 200′ may include a first portion 255, 255′ a second portion 260, 260′ and a third portion 265, 265′. The first portion 255, 255′ may be configured to receive and retain the second end 105b of the tubing 105 in any manner including as previously described above. In some embodiments, the first portion 255, 255′ may be substantially cylindrical, and may extend along longitudinal axis 202, 202′ from the first end 210, 210′ to the second end 215, 215′. During priming, a fluid, e.g., air content and/or liquid, may be flowable in a direction indicated by arrow “D.”

As shown in FIG. 2A, the second end 215 of the connector cap 200 may include the second portion 260. The second portion 260 may extend, from a first end 260a, of the first portion 255 of the connector cap 200 along an axis 203. For example, the second portion 260 may be substantially perpendicular, e.g., 90°±10°, relative to the first portion 255. The second portion 260 may extend radially outward from the longitudinal axis 202. The third portion 265 may extend from a second end 260b of the second portion 260. The third portion 265 may be arranged and configured as a reservoir, and may extend along axis 204 from the second portion 260. For example, the third portion 265 may be substantially perpendicular, e.g., 90°±10°, relative to the second portion 260. Thus arranged, the third portion 265 may be substantially parallel to the first portion 255 but offset therefrom.

During priming, fluid may flow in the direction indicated by arrow “D” into the first portion 255 of the connector cap 200 as described above. Air content may escape from the vent 220, which may be formed as a circular hole such as a pinhole, or any other shape to allow for air content to escape. When a liquid (e.g., dialysate) enters the connector cap 200 the liquid may flow in the direction of arrow “D” to the second end 215 of the connector cap 200. When the liquid rises to the second end 215 the liquid may flow into the second portion 260 in a direction indicated by arrow “E.” The liquid may then fall into the reservoir of the third portion 265 via gravity, to capture excess liquid in a direction indicated by arrow “F.”

FIG. 2A is an exemplary embodiment of a reservoir arranged and configured as a single arm or spoke. FIG. 2B illustrates a reservoir extending circumferentially around a first portion 255′. For example, a second portion 260′ may extend radially from the first portion 255′, so that liquid may flow radially outward from the first portion 255′, e.g., outward from the axis 202′ in a direction indicated by arrows “G.” The liquid may flow into a third portion 265′ formed as a reservoir via gravity to capture excess liquid in a direction indicated by arrows “F.”

Presence of liquid in the reservoir may indicate to a user that priming is complete, so that the treatment may be performed. In embodiments, the reservoir may be sized to receive a predetermined volume of liquid. For example, a volume of liquid needed to fully prime the tubing 105 may be known and the dialysis machine may monitor the liquid delivered during the priming. Priming may include a buffer volume to ensure the tubing is fully primed, accounting for size variations in the tubing. For example, tolerances may result in liquid variations of approximately 3 mL to 20 mL. The volume of liquid delivered into the tubing during priming may take into account these tolerances, which may result in excess liquid being collected in the reservoir. When priming is complete, the connector cap 200, 200′ may be decoupled from the second end 105b of the tubing 105, including the collected excess liquid in the reservoir, and may be discarded.

In some embodiments, the connector cap 200, 200′ may include an absorbent material in the reservoir, so that the liquid may be absorbed during collection. Absorbed liquid may minimize leaks and/or spills after priming when the connector cap 200, 200′ is decoupled from the tubing 105. In some embodiments, the connector cap 200, 200′ may include an additional sealing mechanism for sealing off the reservoir to minimize spillage of the excess liquid.

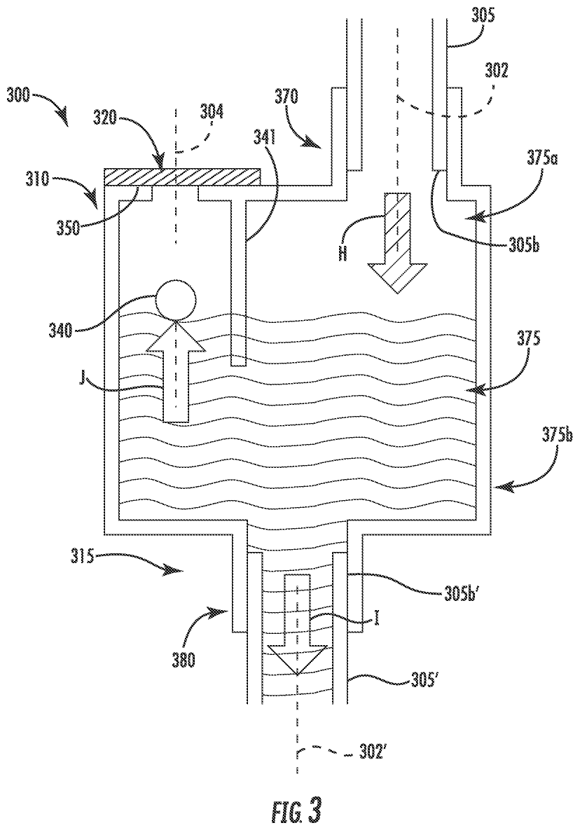

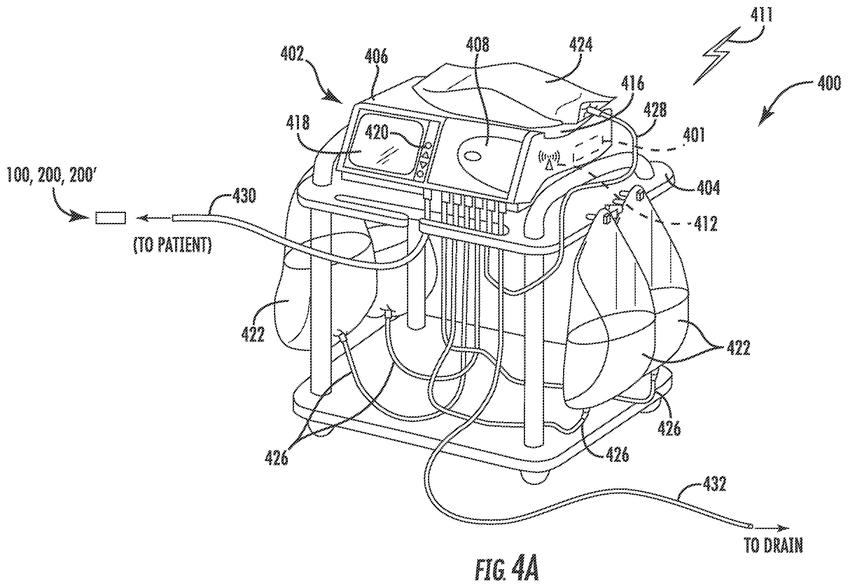

Referring now to FIG. 3, an alternate exemplary embodiment of an air management device 300 for use in a hemodialysis system in accordance with the present disclosure is shown. In connection with the hemodialysis system, the air management device 300 may be referred to as an air managed drip chamber. In use, the air managed drip chamber functions substantially similar to the connector cap 100 described above, however, the air managed drip chamber 300 operates in-line during the hemodialysis operation. Thus arranged, the air managed drip chamber 300 filters air out of the patient's blood as the patient's blood is being removed and reintroduced into the patient's body. This is in contrast to the connector caps 100, 200, 200′ previously described, which are used to prime the line before commencement of the peritoneal dialysis operation. In one embodiment, as shown, the air managed drip chamber 300 may be formed as a hollow body having a first end 310 and a second end 315. During use, the drip chamber 300 may be removably coupled to a plurality of tubing lines to accommodate the fluid flow (e.g., blood flow, dialysate, etc.) during the dialysis operation. As shown, the drip chamber 300 may be removably coupleable to a first tubing 305 and a second tubing 305′. A second end 305b of the first tubing 305 may be insertable at the first end 310 of the drip chamber 300, and an end 305b′ of the second tubing 305′ may be insertable at the second end 315 of the drip chamber 300. The drip chamber 300 may be positioned vertically, e.g., along axis 302, so that the first end 310 is vertically above the second end 315.

The drip chamber 300 may be formed as hollow body for receiving a fluid (e.g., air content and/or liquid such as blood). The first tubing 305 may be attachable to a substantially first cylindrical portion 370 of the drip chamber 300 at the first end 310, which may extend along axis 302. A fluid flow may flow in a direction indicated by arrow “H,” e.g., vertically downward. The fluid flow may flow into a chamber 375 from the first cylindrical portion 370. The chamber 375 may be formed substantially as a cylindrical body to receive a liquid, although the chamber 375 may be any shape to define a cavity. In some embodiments, the chamber 375 may include a curvature so that a liquid may continuously flow from the first cylindrical portion 370 through the chamber 375 and into a second cylindrical portion 380, which may be removably coupled to the second tubing 305′.

When the fluid is a liquid, e.g., dialysate, blood, and/or other body fluids, the liquid may fall via gravity from an upper portion 375a of the chamber 375 to a lower portion 375b of the chamber 375. The liquid may flow out of the chamber 375 into the second substantially cylindrical portion 380 extending along axis 302′ from the lower portion 375b of the chamber 375 at the second end 315. The second tubing 305′ may be coupleable to the second cylindrical portion 380, so that liquid may flow out of the drip chamber 300 in a direction indicated by arrow “I.” In some embodiments, the axes 302, 302′ may be coaxial, e.g., the first and second cylindrical portions 370, 380 may be in alignment with each other, although in other embodiments, the first and second cylindrical portions 370, 380 may be offset from each other so that the axes 302, 302′ may be substantially parallel to each other.

When the fluid includes an air content, the air content may escape from the first end 310 of the drip chamber 300. For example, a vent 320 may be disposed in the first end 310 of the drip chamber 300, e.g., at the upper portion 375a of the chamber 375, e.g., aligned along axis 304. The axis 304 may be substantially parallel to the axes 302, 302′. The vent 320 may be formed similarly to the vent 120 as described above with respect to FIGS. 1A-1D. If the fluid includes both a liquid (e.g., dialysate, blood) and air content, the liquid may flow via gravity in the direction indicated by arrow “H,” while the air content may escape from the vent 320 formed in the drip chamber 300, as indicated by arrow “J.”

The drip chamber 300 may include a movable element 340, which may be similar to the movable element 140 as described above with respect to FIGS. 1A-1D, and may be freely movable within the chamber 375. The drip chamber 300 may also include an internal partition 341 for creating a contained channel for the moveable element 340. For example, as shown, the internal partition 341 may be parallel but offset from an interior surface of a wall of the drip chamber 300. In this manner, the movable element 340 is laterally constrained but freely movable in a vertical direction relative to the vent 320. The movable element 340 may be formed of a buoyant material to float on the liquid that may collect in the chamber 375. When the liquid collects in the chamber 375, the movable element 340 may be moved to the upper portion 375a of the chamber 375, and may be configured to seal the vent 320 so that liquid may not escape through the vent 320. In some embodiments, for example, when a partition is not incorporated, the movable element 340 may be sized to be larger than the first and second cylindrical portions 370, 380, so that the movable element 340 may not obstruct fluid flow from the first and second cylindrical portions 370, 380

Additionally, and/or alternatively, a filter 350 may be included with the drip chamber 300. In one embodiment, the filter 350 may be positioned on the outside of the drip chamber 300 adjacent to the vent 320, although it is envisioned that the filter 350 may be positioned elsewhere including, for example, within the vent 320. The filter 350 may be a hydrophobic filter, and may minimize and/or prevent a liquid from entering and/or exiting the chamber 375 of the drip chamber 300 via the vent 320.

By positioning the vent 320 and optional filter 350 at the upper end 310 and separate from where the blood may enter the chamber 375 via the first tubing 305 and exit the chamber 375 via the second tubing 305′, the filter may remain substantially clear of clotted blood and/or other dried material that may clog the vent 320. The blood and other material may flow through the chamber 375 by gravity while allowing air content to escape through the vent 320 unobstructed.

Referring now to FIGS. 4A and 4B, an example of a dialysis system 400 (e.g., a peritoneal dialysis (PD) system) that is configured in accordance with an exemplary embodiment of the system described herein is shown. In some implementations, the dialysis system 400 may be configured for use at a patient's home (e.g., a home PD system). The dialysis system 400 may include a dialysis machine 402 (e.g., a peritoneal dialysis machine 402, also referred to as a PD cycler) and in some embodiments the machine may be seated on a cart 404. Although the dialysis system 400 is described and illustrated in connection with the dialysis machine 402, in other embodiments, other dialysis machines, may be included in or used in connection with the dialysis system 400 (see FIG. 5).

The dialysis machine 402 may include a housing 406, a door 408, and a cartridge interface including piston assemblies 442, 444 coupled to pump heads 446, 448 for contacting a disposable cassette, or cartridge 434, where the cartridge 434 is located within a compartment 436 formed between the cartridge interface and the closed door 408, and which align with pump chambers 452, 454 formed in the cartridge 434. Fluid lines, or tubing 426, 428, 432, may be coupled to the cartridge 434, and may further include valves for controlling fluid flow to and from fluid bags including fresh dialysate and warming fluid. In another embodiment, at least a portion of the fluid lines may be integral to the cartridge 434. Prior to operation, a user may open the door 408 to insert a fresh cartridge 434, and to remove the used cartridge 434 after operation.

The cartridge 434 may be placed in the compartment 436 of the machine 402 for operation. During operation, dialysate fluid may be flowed into a patient's abdomen via the cartridge 434, and spent dialysate, waste, and/or excess fluid may be removed from the patient's abdomen via the cartridge 434. The door 408 may be securely closed to the machine 402. Peritoneal dialysis for a patient may include a total treatment of approximately 10 to 30 liters of fluid, where approximately 2 liters of dialysate fluid are pumped into a patient's abdomen, held for a period of time, e.g., about an hour, and then pumped out of the patient. This may be repeated until the full treatment volume is achieved, and usually occurs overnight while a patient sleeps. The dialysis machine 402 may also include a user interface such as a touch screen 418 and control panel 420 operable by a user (e.g., a caregiver or a patient) to allow, for example, set up, initiation, and/or termination of a dialysis treatment. The touch screen 418 and the control panel 420 may allow an operator to input various treatment parameters to the dialysis machine 402 and to otherwise control the dialysis machine 402. In addition, the touch screen 418 may serve as a display. The touch screen 418 may function to provide information to the patient and the operator of the dialysis system 400. For example, the touch screen 418 may display information related to a dialysis treatment to be applied to the patient, including information related to a prescription.

Dialysate bags 422 may be suspended from hooks on the sides of the cart 404. Hanging the dialysate bags 422 may improve air management as air content may be disposed by gravity to a top portion of the dialysate bag 422. Although four dialysate bags 422 are illustrated in FIG. 4A, any number “n” of dialysate bags may be connectable to the dialysis machine 402 (e.g., 1 to 5 bags, or more), and reference made to first and second bags is not limiting to the total number of bags used in a dialysis system 400. For example, the dialysis machine may have dialysate bags 422a, . . . 422n connectable in the system 400. In some embodiments, connectors and tubing ports may connect the dialysate bags 422 and lines for transferring dialysate.

The dialysis machine 402 may include a processing module 401 that resides inside the dialysis machine 402, the processing module 401 being configured to communicate with the touch screen 418 and the control panel 420. The dialysis machine 402 may be configured to connect to a network 411. The connection to network 411 may be via a wired and/or wireless connection. The dialysis machine 402 may include a connectivity component 412 configured to facilitate the connection to the network 411.

In some embodiments, a heater tray 416 may be positioned on top of the housing 406. The heater tray 416 may be any size and shape to accommodate a bag of dialysate (e.g., a 5L bag of dialysate) for batch heating. In some embodiments, the heater tray 416 may include a heating element 440, for heating the dialysate prior to delivery into the patient. A heater bag 424 may be positioned in the heater tray 416. Dialysate from the dialysate bags 422 may be transferred to the heater bag 424 in batches. For example, a batch of dialysate may be transferred from the dialysate bags 422 to the heater bag 424, where the dialysate is heated by the heating element 440. When the batch of dialysate has reached a predetermined temperature (e.g., approximately 98°−100° F., 37° C.), the dialysate may be flowed into the patient.

The dialysate bags 422 and the heater bag 424 may be connected to the cartridge 434 via dialysate bag lines or tubing 426 and a heater bag line or tubing 428, respectively. The dialysate bag lines 426 may be used to pass dialysate from dialysate bags 422 to the cartridge during use, and the heater bag line 428 may be used to pass dialysate back and forth between the cartridge and the heater bag 424 during use. A drain line 432 may be connected to the cartridge 434. The drain line 432 may be connected to a drain or drain receptacle and may be used to pass dialysate from the cartridge to the drain or drain receptacle during use.

FIG. 5 illustrates an exemplary embodiment of a dialysis machine 500 in accordance with the present disclosure. The dialysis machine 500 may be implemented in the dialysis system 400 and may include, for example, a housing 506, a processing module 501, a connection component 512, a touch screen 518, and a control panel 520 operable by a user (e.g., a caregiver or a patient) to allow, for example, set up, initiation, and/or termination of a dialysis treatment.

The touch screen 518 and the control panel 520 may allow a user to input various treatment parameters to the dialysis machine 500 and to otherwise control the dialysis machine 500. In addition, the touch screen 518 may serve as a display. The touch screen 518 may function to provide information to the patient and the operator of the dialysis system 500. For example, the touch screen 518 may display information related to a dialysis treatment to be applied to the patient, including information related to a prescription.

The dialysis machine 500 may include a processing module 501 that resides inside the dialysis machine 500, the processing module 501 being configured to communicate with the touch screen 518 and the control panel 520. The processing module 501 may be configured to receive data from the touch screen 518, the control panel 520 and sensors, e.g., air, temperature and pressure sensors, and control the dialysis machine 500 based on the received data. For example, the processing module 501 may adjust the operating parameters of the dialysis machine 500.

The dialysis machine 500 may be configured to connect to a network. The connection to network may be via a wired and/or wireless connection. The dialysis machine 500 may include a connectivity component 512 configured to facilitate the connection to the network. The connectivity component 512 may be a transceiver for wireless connections and/or other signal processor for processing signals transmitted and received over a wired connection. Other medical devices (e.g., other dialysis machines) or components may be configured to connect to the network and communicate with the dialysis machine 500. The processing module 501 and the connectivity component 512 may be configured similarly to the processing module 401 and connectivity component 412 described above.

One or more heating elements may be disposed internal to the dialysis machine 500. For example, a warmer pouch 524 may be insertable into an opening 510 in a direction indicated at arrow 514. It is also understood that the warmer pouch 524 may be connectable to the dialysis machine 500 via tubing, or fluid lines, via a cartridge. The tubing may be connectable so that dialysate may flow from containers (e.g., dialysate bags), through the warmer pouch 524 for heating, and to the patient.

In such in-line heating embodiments, the warmer pouch 524 may be configured so dialysate may continually flow through the warmer pouch to achieve a predetermined temperature before flowing into the patient. Internal heating elements (not shown) may be positioned above and/or below the opening 510, so that when the warmer pouch 524 is inserted into the opening 510, the one or more heating elements may affect the temperature of dialysate flowing through the warmer pouch 524. In some embodiments, the internal warmer pouch may instead be a portion of tubing in the system that is passed by, around, or otherwise configured with respect to, a heating element(s). It is understood that FIG. 5 illustrates dialysate continuously flowing through the warmer pouch 524 “in-line” with the dialysis machine 500, reaching an acceptable temperature by the application of internal heating elements, and that FIGS. 4A, 4B, as described above, illustrate that dialysate may be transferable to and stored in the heater bag 424 by “batch” until reaching an acceptable temperature for use.

A patient line, e.g., a patient line 430 as shown in FIG. 4A, may be connected to a cartridge. The patient line may be tubing 105 as described above with respect to FIGS. 1A-1D, 2A, and 2B, and a connector cap 100, 200, 200′ may be attachable to at least an end of the patient line during a priming step prior to a dialysis treatment. Additional features and descriptions related to the connector caps described herein may also be included in the dialysis machine 402, 500. The connector cap 100, 200, 200′ may be configured for a peritoneal dialysis treatment, e.g., when dialysate is flowed through the patient line to purge air content prior to performing a treatment. When the priming step is complete, the connector cap 100, 200, 200′ may be removed from the end of the patient line, and the patient line may be connected to the patient, e.g., via a catheter, and to pass dialysate back and forth between the cartridge and the patient's peritoneal cavity.

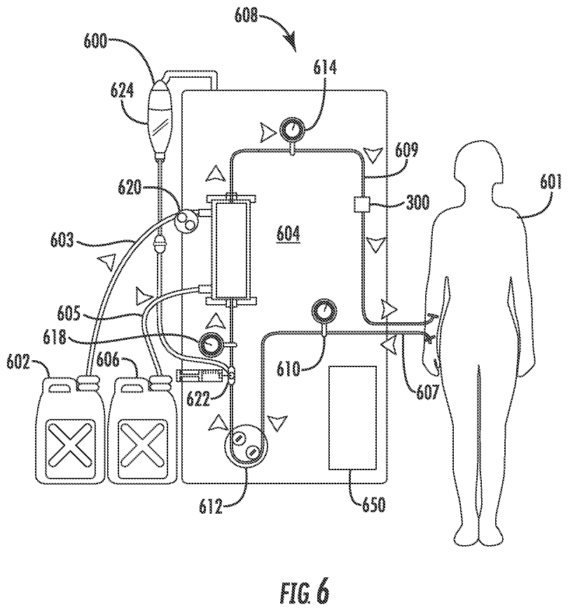

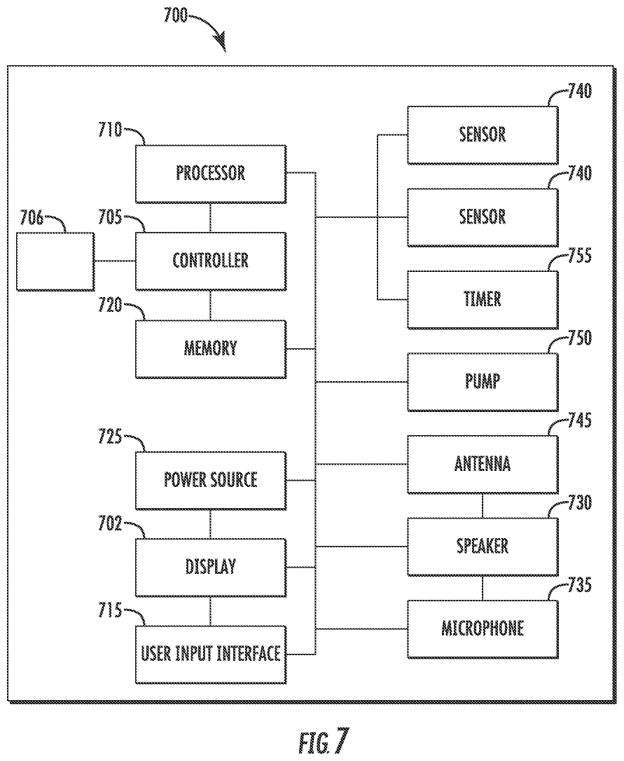

Referring now to FIG. 6, a diagram of an exemplary embodiment of a dialysis system 600 in accordance with the present disclosure is shown. The dialysis system 600 may be configured to provide hemodialysis treatment to a patient 601 by a dialysis machine 608. A fluid reservoir 602 may deliver fresh dialysate to a dialyzer 604 via tubing 603, and reservoir 606 may receive spent dialysate once it has passed through the dialyzer 604 via tubing 605. A hemodialysis operation may filter particulates and/or contaminates from a patient's blood through a patient external filtration device, for example, a dialyzer 604. As the dialysate is passed through the dialyzer 604, so too unfiltered patient blood may be passed into the dialyzer via tubing 607 and filtered blood may be returned to the patient via tubing 609. Arterial pressure may be monitored via pressure sensor 610, inflow pressure monitored via sensor 618, and venous pressure monitored via pressure sensor 614. An air managed drip chamber 300 as previously described herein may be utilized to ensure that air is not introduced into patient blood as it is filtered and returned to the patient 601. The flow of blood and the flow of dialysate are controlled via respective pumps, including a blood pump 612 and a fluid pump 620. Heparin 622, a blood thinner, may be used in conjunction with saline 624 to ensure blood clots do not form or occlude blood flow through the system.

In some embodiments, the treatment system 600 may include a controller 650. The controller 650 may be configured to monitor fluid pressure readings to identify fluctuations indicative of patient parameters, such as heart rate or respiration rate, or both. The controller 650 may also be operatively connected to and/or communicate with additional sensors or sensor systems, although the controller 650 may use any of the data available.

Referring now to FIG. 7, a schematic of an exemplary embodiment of a dialysis machine 700 and a controller 705 in accordance with the present disclosure are shown. The dialysis machine 700 may be a dialysis machine, e.g., a peritoneal dialysis machine, and/or a hemodialysis machine, for performing a dialysis treatment on a patient, and may be included in the systems 400, 600 for dialysis machines 402, 500, 608, described above. Additionally, components described with respect to the dialysis machine 700 may also be included in the dialysis machines 402, 500, 608. It is understood that the dialysis machine 700 may be dialysis machines 402, 500, 608, and/or may include any or all of the features of dialysis machines 402, 500, 608. A power source 725 may provide power and/or a connection to an external power source to the dialysis machine 402, 500, 608.

The controller 705 may automatically control execution of a treatment function during a course of dialysis treatment. For example, the controller 705 may control the delivery and transfer of dialysate for dialysis machines 402, 500, 608. The controller 705 may be operatively connected to one or more sensors 740 and may deliver one or more signals to execute one or more treatment functions, e.g., including a priming step. For example, dialysis treatment may include transferring dialysate from the dialysate bag 422 through the patient line when the connector cap 100, 200, 200′ is attached to the end of the tubing. The sensors 740 may trigger an alert to a user, e.g., when a priming step has completed, to minimize liquid overflow. The sensors 740 may be operatively connectable to an I/O board in communication with the processor 710, such that signals may be sent to a LED or other alerting function. As described above, the alert may be audible, such as a noise issued from a speaker 730, and/or visual, including an LED, a notification on the touch screen, or both.

In some embodiments, a timer 755 may be included for timing triggering of sensors 740. It is understood that sensors, including but not limited to pressure sensors, weight sensors, flow sensors, air sensors, and temperature sensors, may detect dialysate temperature, fluid volume, fluid flow rate, and fluid flow pressure for the dialysis machine 402, 500, 608 to determine flow delivery, including priming of the tubing. For example, the dialysis machine 402, 500, 608 may include a plurality of sensors for detection and/or measurement of any combination of temperature, pressure, volume, fluid flow. Multiple sensors may also be included to detect and/or measure individually the temperature, pressure, volume, fluid flow. Although FIG. 7 illustrates the components integral to the dialysis machine 700, at least one of the controller 705, processor 710, and/or memory 720 may be configured to be external and wired or wirelessly connected to the dialysis machine 402, 500, 608, as an individual component of a dialysis system. In some embodiments the controller 705, processor 710 and memory 720 may be remote to the dialysis machine and configured to communicate wirelessly.

In some embodiments, the controller 705, processor 710, and/or memory 720 of the dialysis machine 700 may receive sensor 740 signals indicating the priming step, as well as process parameters, such as temperature, pressure, volume, flow rate, and the like. The controller 705 may also detect connection of all dialysate bags 422 connected.

Communication between the controller 705 and the system may be bi-directional, whereby the system acknowledges control signals, and/or may provide state information associated with the system and/or requested operations. For example, system state information may include a state associated with specific operations to be executed by the system (e.g., trigger pump to deliver dialysate, trigger pumps and/or compressors to deliver filtered blood, and the like) and a status associated with specific operations (e.g., ready to execute, executing, completed, successfully completed, queued for execution, waiting for control signal, and the like).

In embodiments, the dialysis machine 402, 500, 608 may include at least one pump 750 operatively connected to the controller 705. During operation, the controller 705 may control the pump 750 for pumping fluid, e.g., fresh and spent dialysate, to and from a patient, and/or to prime the patient line tubing. The pump 750 may also pump dialysate from the dialysate bag 422 to the heater bag 424. In embodiments where the warmer pouch 524 is in-line with the dialysis machine 500, the pump 750 may pump the dialysate through the warmer pouch 524 directly to the patient line. The controller 705 may also be operatively connected to a speaker 730 and a microphone 735 disposed in the dialysis machine 402, 500, 608, e.g., for generating audible alerts and/or alarms.

A user input interface 715 may include a combination of hardware and software components that allow the controller 705 to communicate with an external entity, such as a patient or other user, and a display 702 may display information to the user or medical professional. These components may be configured to receive information from actions such as physical movement or gestures and verbal intonation. In embodiments, the components of the user input interface 715 may provide information to external entities. Examples of the components that may be employed within the user input interface 715 include keypads, buttons, microphones, touch screens, gesture recognition devices, display screens, and speakers. The dialysis machine 402, 500, 608 may also be wirelessly connectable via the antenna 745 for remote communication.

Sensors 740 may be included for monitoring one or more parameters, including monitoring a priming operation, may be operatively connected to at least the controller 705, processor 710, and memory 720. Sensors 740 may include a pressure sensor for monitoring fluid pressure of the dialysis machine 402, 500, 608, although the sensors 740 may also include any of a heart rate sensor, a respiration sensor, a temperature sensor, a flow sensor, a weight sensor, a video sensor, an air sensor, an air bubble sensor, a thermal imaging sensor, an electroencephalogram sensor, a motion sensor, audio sensor, an accelerometer, and/or capacitance sensor. It is appreciated that the sensors 740 may include sensors with varying sampling rates, including wireless sensors.

The processor 710 may be configured to execute an operating system, which may provide platform services to application software, e.g., for operating the dialysis machine 402, 500, 608. These platform services may include inter-process and network communication, file system management and standard database manipulation. One or more of many operating systems may be used, and examples are not limited to any particular operating system or operating system characteristic. In some examples, the processor 710 may be configured to execute a real-time operating system (RTOS), such as RT/Linux, or a non-real time operating system, such as BSD or GNU/Linux. As described above, it is also understood that the processor 710 may be operatively connected to an I/O board, for communication between the sensors 740 and a LED or other alerting function.

According to a variety of examples, the processor 710 may be a commercially available processor such as a processor manufactured by INTEL, AMD, MOTOROLA, and FREESCALE. However, the processor 710 may be any type of processor, multiprocessor or controller, whether commercially available or specially manufactured. For instance, according to one example, the processor 710 may include an MPC823 microprocessor manufactured by MOTOROLA.

The memory 720 may include a computer readable and writeable nonvolatile data storage medium configured to store non-transitory instructions and data. In addition, the memory 720 may include a processor memory that stores data during operation of the processor 710. In some examples, the processor memory includes a relatively high performance, volatile, random access memory such as dynamic random access memory (DRAM), static memory (SRAM), or synchronous DRAM. However, the processor memory may include any device for storing data, such as a non-volatile memory, with sufficient throughput and storage capacity to support the functions described herein. Further, examples are not limited to a particular memory, memory system, or data storage system.

The instructions stored on the memory 720 may include executable programs or other code that may be executed by the processor 710. The instructions may be persistently stored as encoded signals, and the instructions may cause the processor 710 to perform the functions described herein. The memory 720 may include information that is recorded, on or in, the medium, and this information may be processed by the processor 710 during execution of instructions. The memory 720 may also include, for example, specification of data records for user timing requirements, timing for priming or treatment and/or other operations, and historic sensor information. The medium may, for example, be optical disk, magnetic disk or flash memory, among others, and may be permanently affixed to, or removable from, the controller 705.

The controller 705 may be disposed in the dialysis machine 402, 500, 608 or may be coupled to the dialysis machine 402, 500, 608 via a communication port or wireless communication links, shown schematically as communication element 706. According to various examples, the communication element 706 may support a variety of one or more standards and protocols, examples of which include USB, Wi-Fi, TCP/IP, Ethernet, Bluetooth, Zigbee, CAN-bus, IP, IPV6, UDP, UTN, HTTP, HTTPS, FTP, SNMP, CDMA, NMEA and/or GSM. As a component disposed within the dialysis machine 700, the controller 705 may be operatively connected to any one or more of the sensors 740, pump 750, or combinations thereof. The controller 705 may communicate control signals or triggering voltages to the components of the dialysis machine 402, 500, 608. As discussed, exemplary embodiments of the controller 705 may include wireless communication interfaces. The controller 705 may detect remote devices to determine if any remote sensors are available to augment any sensor data being used to evaluate the patient.

Some embodiments of the disclosed system may be implemented, for example, using a storage medium, a computer-readable medium or an article of manufacture which may store an instruction or a set of instructions that, if executed by a machine (i.e., processor or microcontroller), may cause the machine to perform a method and/or operation in accordance with embodiments of the disclosure. In addition, a server or database server may include machine readable media configured to store machine executable program instructions. Such a machine may include, for example, any suitable processing platform, computing platform, computing device, processing device, computing system, processing system, computer, processor, or the like, and may be implemented using any suitable combination of hardware, software, firmware, or a combination thereof and utilized in systems, subsystems, components, or sub-components thereof. The computer-readable medium or article may include, for example, any suitable type of memory unit, memory device, memory article, memory medium, storage device, storage article, storage medium and/or storage unit, for example, memory (including non-transitory memory), removable or non-removable media, erasable or non-erasable media, writeable or re-writeable media, digital or analog media, hard disk, floppy disk, Compact Disk Read Only Memory (CD-ROM), Compact Disk Recordable (CD-R), Compact Disk Rewriteable (CD-RW), optical disk, magnetic media, magneto-optical media, removable memory cards or disks, various types of Digital Versatile Disk (DVD), a tape, a cassette, or the like. The instructions may include any suitable type of code, such as source code, compiled code, interpreted code, executable code, static code, dynamic code, encrypted code, and the like, implemented using any suitable high-level, low-level, object-oriented, visual, compiled and/or interpreted programming language.

As used herein, an element or operation recited in the singular and proceeded with the word “a” or “an” should be understood as not excluding plural elements or operations, unless such exclusion is explicitly recited. Furthermore, references to “one embodiment” of the present disclosure are not intended to be interpreted as excluding the existence of additional embodiments that also incorporate the recited features.

While the systems and techniques described herein for priming have been largely explained with reference to a dialysis machine, in particular, a peritoneal dialysis machine, the systems and techniques described for priming may be used in connection with other types of medical treatment systems and/or machines, such as a hemodialysis machine or other medical treatment device involving medical fluids. In some implementations, the dialysis machine may be configured for use in a patient's home (e.g., a home dialysis machine). The home dialysis machine can take the form of a peritoneal dialysis machine or a home hemodialysis machine.

The present disclosure is not to be limited in scope by the specific embodiments described herein. Indeed, other various embodiments of and modifications to the present disclosure, in addition to those described herein, will be apparent to those of ordinary skill in the art from the foregoing description and accompanying drawings. Thus, such other embodiments and modifications are intended to fall within the scope of the present disclosure. Furthermore, although the present disclosure has been described herein in the context of a particular implementation in a particular environment for a particular purpose, those of ordinary skill in the art will recognize that its usefulness is not limited thereto and that the present disclosure may be beneficially implemented in any number of environments for any number of purposes. Accordingly, the claims set forth below should be construed in view of the full breadth and spirit of the present disclosure as described herein.

What is claimed is:

1. A dialysis system for conducting a dialysis treatment, comprising:
a dialysis machine for transferring a liquid to a patient via tubing; and
a connector cap removably coupled to an end of the tubing, the connector cap being arranged and configured to filter out air content prior to transferring the liquid to the patient.

2. The dialysis system according to claim 1, wherein the connector cap includes a first open end and a second end, the second end including a vent.

3. The dialysis system according to claim 2, wherein the end of the tubing is insertable into the first open end of the connector cap, the air content being filterable out of the vent of the second end of the connector cap.

4. The dialysis system according to claim 2, wherein the connector cap includes a hydrophobic filter disposed at the second end of the connector cap, the air content being filterable out of the vent through the hydrophobic filter.

5. The dialysis system according to claim 2, wherein the connector cap includes a circumferential flange extending from an inner surface of the connector cap, the flange including an aperture formed therein, the aperture defining a fluid flow path for the liquid, or air content, or both, from the first end to the second end through the connector cap.

6. The dialysis system according to claim 5, wherein the connector cap includes a movable element, the movable element being movably positioned between the flange and the second end of the connector cap.

7. The dialysis system according to claim 6, wherein the movable element allows passage of the air content flowing through the connector cap.

8. The dialysis system according to claim 6, wherein in response to the liquid entering the connector cap, the movable element is movable by the liquid to seal the vent.

9. The dialysis system according to claim 1, wherein the connector cap includes a reservoir for receiving the liquid.

10. The dialysis system according to claim 1, wherein the dialysis machine is a peritoneal dialysis machine.

11. The dialysis system according to claim 10, wherein the liquid is a dialysate solution.

12. A method of conducting a dialysis treatment, comprising:
transferring a liquid to a patient via tubing by a dialysis machine;
removably coupling a connector cap to an end of the tubing, the connector cap including a vent; and
filtering out air content via the vent of the connector cap prior to transferring the liquid to the patient.

13. The method according to claim 12, wherein the connector cap includes an open first end and a second end, the vent being disposed in the second end.

14. The method according to claim 12, wherein the connector cap includes a hydrophobic filter disposed at the second end of the connector cap, such that the air content is filtered out of the vent through the hydrophobic filter.

15. The method according to claim 12, wherein the connector cap includes:
a circumferential flange extending from an inner surface of the connector cap, the flange including an aperture defining a fluid flow path; and
a movable element movably positioned between the flange and the second end of the connector cap;
wherein, in response to a liquid flowing into the connector cap, sealing the vent by the movable element.

16. A connector cap for a dialysis system, comprising:
a hollow body having a first open end and a second end, the second end having a vent;
a flange extending inward from an inner surface of the hollow body; and
a movable element disposed in the second end of the hollow body, the movable element being movable positioned between the flange and the second end of the hollow body.

17. The connector cap according to claim 16, wherein tubing is insertable in the first open end of the hollow body of the connector cap.

18. The connector cap according to claim 17, further comprising a reservoir extending from the hollow body, such that the reservoir is substantially parallel to the tubing.

19. The connector cap according to claim 16, wherein the dialysis system is a peritoneal dialysis system.

20. The connector cap according to claim 16, wherein the moveable element is configured to move within the connector cap to seal the vent in the second end of the hollow body, or to seal an aperture in the flange, depending on the flow path of fluid through the connector cap.

21. The connector cap according to claim 20, wherein the fluid is liquid, or air content, or both.

22. A dialysis system for conducting a dialysis treatment, comprising:
a dialysis machine for circulating a liquid including blood into and out of a patient via tubing; and
an air managed drip chamber coupled to an end of a first tubing for receiving the liquid and a second tubing for transferring the liquid to the patient, the drip chamber including a body having a cavity for receiving the liquid therein, a vent for releasing air content from the cavity prior to transferring the liquid to the patient; and a movable element positioned within the cavity, the movable element being arranged and configured to seal the vent when an amount of liquid is received within the cavity.

23. The dialysis system according to claim 22, wherein the drip chamber includes a body having a first end and a second end, the first tubing being receiving within the first end of the body, the second tubing being received within the second end of the body, and the vent is formed in the first end of the body.

24. The dialysis system according to claim 22, wherein the body includes an internal partition extending into the cavity for creating a contained channel for the moveable element.

25. The dialysis system according to claim 22, wherein the movable element is arranged and configured to float on the liquid received within the cavity, the moveable element contacting and sealing the vent when a sufficient amount of liquid is received within the cavity.
